(19) United States
(12) Patent Application Publication
(10) Pub. No.: US 2008/0224051 A1
(43) Pub. Date: Sep. 18, 2008

(54) Multi-axis moveable gantry gamma camera

(75) Inventor: Richard Conwell, Del Mar, CA (US)

Correspondence Address: Law Office of Scott C Harris Inc, PO Box 1389, Rancho Santa Fe, CA, 92067, US

(73) Assignee: DIGIRAD CORPORATION, Poway, CA (US)

(21) Appl. No.: 12/048,172

(22) Filed: Mar. 13, 2008

Related U.S. Application Data
(60) Provisional application No. 60/894,856, filed on Mar. 14, 2007.

(51) Int. Cl. G01T 1/166; A61B 6/00

(52) U.S. Cl. 250/363.08; 250/363.02; 250/363.1; 250/370.09; 600/436

(57) ABSTRACT

A multiaxis gantry camera has additional axes of movement that allow unexpected functions. A first function allowed the collimator is to be exchanged while maintaining them in a configuration that saves space. The device can also be used to calibrate in a space-saving configuration. Different turning configurations can be allowed to change the orientation over which scanning is conducted.

This application claims priority from U.S. Provisional application No. 60/894,856, filed Mar. 14, 2007, the disclosure of which is herewith incorporated by reference.

### BACKGROUND

Gamma camera gantry systems have been developed that utilize robotic, multi-axes, rectilinear or polar coordinate actuators to move the imaging detectors. Programmable devices control these actuators along many different paths of detector motion. These gantries often have one or more vertical supports to which some of the actuators are attached, and moving parts that allow one or more types of detector motion.

Because gamma rays cannot be reflected or refracted, gamma cameras use collimator devices to limit the direction and area from which gamma rays can enter the camera's imaging detector. Collimators are typically made from high Z-materials such as lead having thousands of small diameter holes through which the gamma rays pass. The diameter and length of the holes define the imaging characteristics of the collimator. The amount of lead between the holes determines the gamma ray energy that can be imaged.

Most gamma cameras have more than one collimator type, with many having more than five types.

Modern gamma cameras have collimators that often weigh several hundred pounds. This weight makes it very difficult for a person to manually remove one type of collimator and replace it with another. In addition, storage of these collimators takes up floor space in the imaging suite.

For some time, gamma cameras have used collimator exchangers to eliminate the need to manually lift the collimators. These exchangers typically use a collimator storage cart that an operator rolls up to the imaging detector. The operator then pulls or pushes the existing collimator from the head to the storage cart. The cart is then rolled away. The new collimator (on another cart) is then rolled to the detector and pushed or pulled onto the detector.

Another way of doing this used an automated system to drive the cart. In time, the manual positioning of the collimator storage cart and the manual removal/installation of the collimator was automated. Regardless, the cart took up valuable floor space; thereby increasing the size of the room required to house the camera system.

Philips' SKYLight system was one of the first large field of view, robotic, multi-axis, programmable gantry gamma cameras. This introduced a new approach to automatic collimator exchange. The detector heads were robotically moved to a location where a wall mounted collimator holder would swing/deploy from a position parallel to the wall, to a position perpendicular to the wall. The robotic gantry then places the currently installed collimator onto the holder and the holder swings back parallel to the wall. The gantry would then travel to a new location where it would pick up a new collimator from a similarly wall mounted, swinging collimator holder.

For dual imaging head systems, each side of the deployed holder could hold a collimator. This approach had the advantage of placing the collimators against the wall thereby reducing the floor space required to store the collimators. However, it has the costly disadvantage of requiring an actuator on each wall mounted, swinging collimator storage holder in order to orient the collimator to be parallel to the imaging detector surface for mounting/de-mounting of the collimator. In addition, placing a collimator on each side of the holder extends the depth of the holder. This extended depth as well as the need to house the actuators could extend the size of the room required to house the camera and collimator exchanger.

In an effort to provide more space to meet the required detector-to-isotopic point source separation distance for calibration, camera systems are sometimes located so that the detector heads can be aligned to face towards a door on the wall opposite the detectors. This allows the isotopic point source to be placed in a hallway gaining the required separation distance.

Imaging a patient in a hospital bed with robotic, multi-axis, programmable gantry gamma camera systems that are floor mounted, has required that the hospital bed be placed across the track that runs along the floor supporting the vertical gantry tower.

### SUMMARY

The present application describes additional movement capability in a gantry camera system, that allows special functions not possible with prior art devices.

More specifically, embodiments disclose a new movement of a 180 degree rotation on a gantry tower, that rotation of the gantry tower, and in a way that allows the camera and heads to be moved away from the patient being imaged.

### BRIEF DESCRIPTION OF THE DRAWINGS

FIG. 1 shows a perspective view of the system of an embodiment with the movable device and the collimator storage devices;

FIG. 2 shows a plan view of the imaging detector over a patient imaging table;

FIG. 3 shows a plan view of the gantry arm moving towards a location where it will put the collimator into the collimator storage device;

FIG. 4 shows rotation of the arm to allow it to interact with the collimator storage device;

FIG. 5 illustrates the collimator device exchange with the storage device;

FIG. 6 shows the collimator device turned backwards for calibration;

FIG. 7 shows an alternative configuration where the patient imaging table has been removed and a patient hospital bed is wheeled under the device.

### DETAILED DESCRIPTION

The inventor found that many of the existing gantry systems have a limited amount of motion.

The inventor recognized that a significant limitation, and source of many different problems, would be solved or reduced if these gantry systems enabled their vertical support to rotate about its vertical axis. This new motion correspondingly allows a decrease in the size of the room housing the camera, and housing its associated optional collimators. The recognition of this problem allowed the inventor to conceive of new features allowed by new movements that are disclosed herein.

The imaging detectors on a gamma camera often require periodic calibration. Some of the intrinsic calibrations require that an isotopic point source be positioned at a distance of five times (5×) the maximum dimension of the detector's field of view. For a 21″×16″ field of view (which is a size of many, but not all, detectors), this distance is 8.75 feet from the face of the detector. Proper calibration requires that there be no obstructions between the isotopic source and the detector.

Robotic, multi-axis, programmable gantry gamma camera systems are often located in a place that requires that the imaging detectors be spaced from the wall. This inability to get close to a wall, combined with the need for the 5× detector size distance from the detector to a location where an isotopic point source can be placed, even further increases the necessary room size. An embodiment addresses this problem by allowing this movement that allows placing the imaging detector against a wall, facing away from the wall.

On occasion, it is necessary to perform imaging procedures on patients that cannot be easily removed from their hospital bed. Often these imaging procedures are whole body scans where it is desirable to have both detectors above the patient and able to scan down the body. This may even further increase the room size.

The inventor realized that a means of enabling a multi-axis, programmable gantry gamma camera system to be housed in a small room while still allowing for an automatic collimator exchanger, the ability to perform intrinsic calibrations with isotopic point sources and the ability to easily perform imaging procedures with a patient in a hospital bed that has been moved to the gamma camera would be useful.

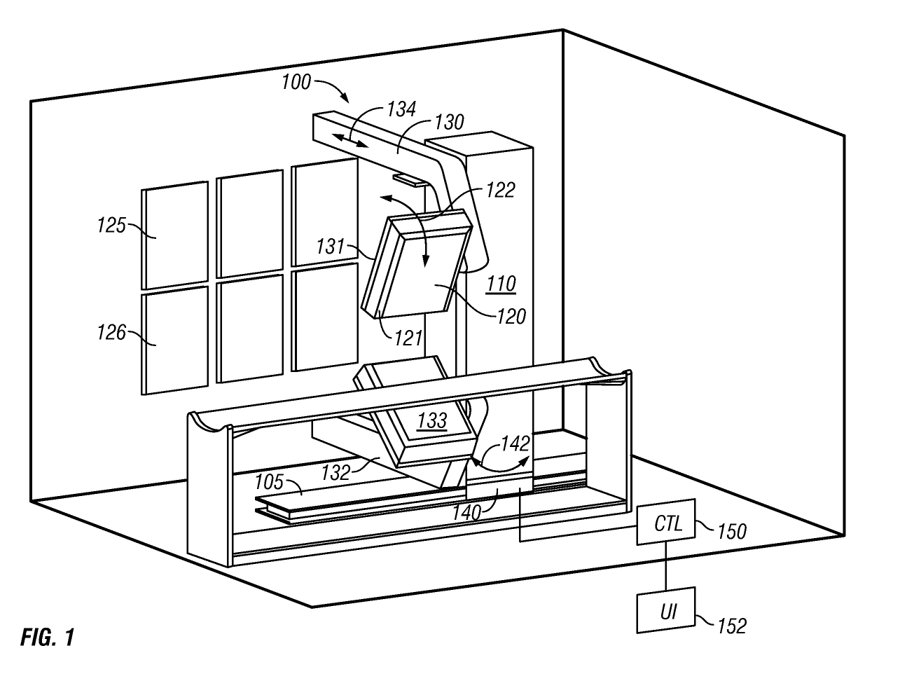

A robotic, multi-axis, gantry gamma camera system 100 is shown in FIG. 1. This may be a floor mounted device that has a gantry tower track 105 that runs along the floor. The vertical gantry tower device 110 runs along this track. This movement enables the imaging parts to move parallel to the patient bed.

In an embodiment, a collimator 120 is placed on the face 121 of the imaging detector. Spare collimators 125 are stored on a wall-mounted rack. However, unlike the prior art, the collimator holders are mounted on the wall, so that the collimator surfaces are flat against the wall (with the collimator holes perpendicular to the wall). These collimator holders are maintained as flat on the wall and do not swing. One embodiment provides only one collimator on each holder. An embodiment shows two collimator holders 125, 126, one vertically above the other. Preferably, there are 6 or more collimator holders as shown.

A rotation stage 140 is actuated to rotate the vertical gantry tower 110, along with everything that is connected to the tower, e.g., its associated detector arm(s) 130, 132 and imaging detectors 131, 133.

When the device is rotated by 90° around the vertical axis, it allows both imaging detectors to be positioned in tandem along the axis of the imaging table, e.g. adjacent a patient hospital bed (above or below the bed) . Doing so enables more of the axial extent of the patient's body to be imaged simultaneously. In addition, orienting the detector heads in this manner enables a patient's hospital bed to be in the same position as was the patient imaging table, parallel to the gantry tower track.

Additional axes of motion can be used with the rotation stage 140 to allow advantages that are not reasonably predictable from the prior art.

An axis-of-motion shown as 122 allows moving the imaging detector surface 121. The surface can be placed parallel to the collimator holder(s) 125 for mounting/de-mounting. This embodiment allows 180° rotation about the system's vertical axis shown by the arrow 142, enabling the imaging detectors to be re-oriented from facing perpendicular to the imaging table axis, to facing perpendicularly away form the table axis and parallel towards the wall mounted collimator storage holder(s) 125.

The addition of this 180° rotation about the system's vertical axis also unexpectedly enables the detectors to be positioned close to one wall, to allow the maximum distance from the imaging surface to the opposite wall.

The gantry arms 130 can also be moved in the direction 134 to allow the motion and functions described herein.

This system allows a number of advantages that provide significant advantages, and advantages which are not reasonably expectable from the prior art.

The addition of the 180° rotation about the robotic, multi-axis, programmable gantry camera system's vertical axis enables each collimator holder to be fixed in place. No actuator is required to swing the collimator(s) into position. This significantly reduces system complexity by eliminating actuators on each collimator holder. Since accurate positioning need not be maintained on a swinging apparatus, complexity and cost is further reduced.

Additionally, because each collimator holder holds only one collimator, the depth of collimator holder system is minimized and the need to enlarge the room is reduced or eliminated.

The addition of this 180° rotation about the robotic, multi-axis, programmable gantry camera system's vertical axis also enables the detectors to be positioned close to one wall so that there is the maximum distance from the imaging surface to the opposite wall. This minimizes the size of the room into which the robotic, multi-axis, programmable gantry gamma camera can be installed while still providing space to perform system calibrations with an isotopic point source.

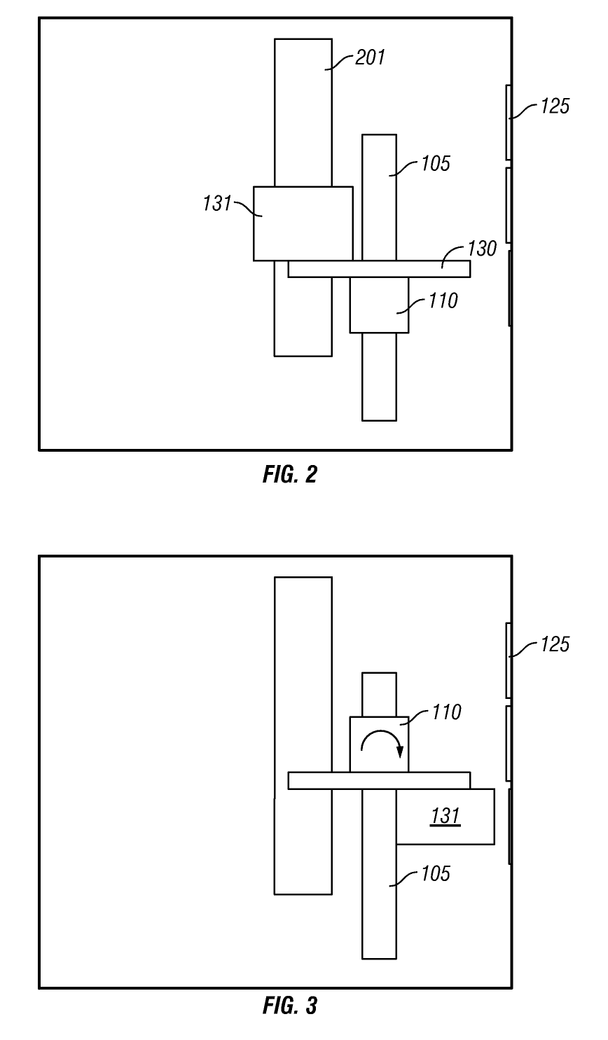

The ability to rotate the vertical gantry by 90° allows whole body hospital bed imaging to be performed without the need to place the hospital bed across the vertical tower gantry track. This avoids the need to lift the bed over the tracks and reduces the risk of accidental collision of the camera system with the patient's hospital bed. The operation is explained with relation to FIGS. 2-6.

FIG. 2 illustrates a plan view from above showing a single imaging detector 131 on the movable gantry arm 130 and how this can be located over a table 201. The gantry tower itself 110 is shown sliding on the track 105. Note that the tower can also rotate perpendicular to the direction of the track. The collimator storage device 125 is shown holding a collimator therein.

FIG. 3 illustrates how the gantry tower can be rotated by 180°. The imaging detector 131 has been moved to the opposite side of the gantry power track 105 as compared with the orientation in FIG. 3. The imaging detector is therefore no longer over the patient table; instead, it is facing the wall housing the collimator holders such as 125.

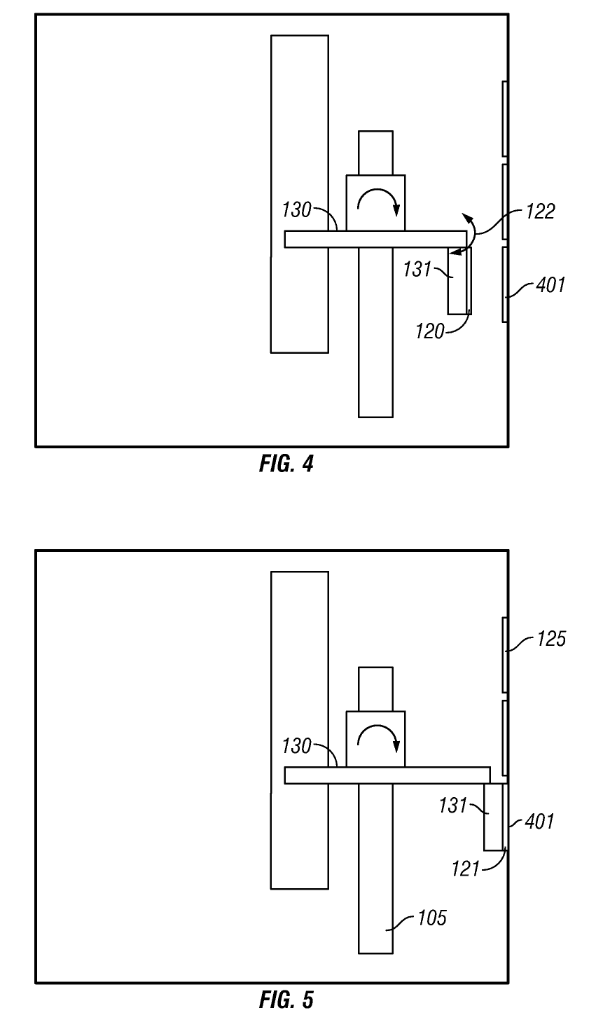

FIG. 4 illustrates how the imaging detector 131 has been rotated so that the imaging surface 120 is now parallel to and facing an empty collimator holder shown as 401. At this point, gantry arm 130 is moved towards the wall and collimator holder 401. This can place the collimator device 120 itself into the collimator holder 401. While in this position, the imaging detector 131 can be moved along the gantry power track 105, for example to use the collimator from a different storage holder 125.

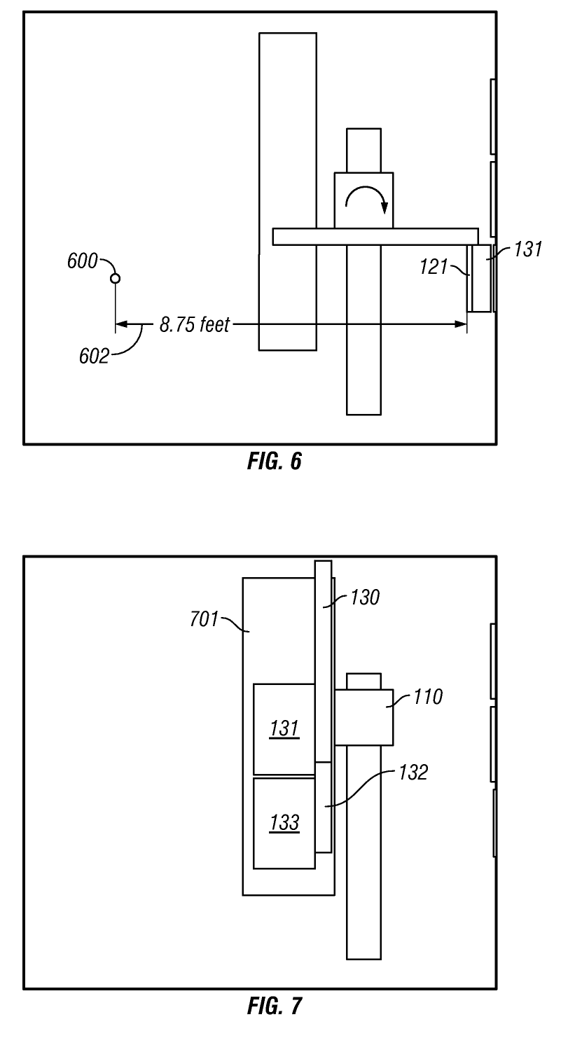

FIG. 6 illustrates the imaging detector 131 being rotated, so that the imaging detector surface 121 is facing away from the wall. The detector surface is now facing the isotopic (global) point source 600 that can be used for system calibration. 602 illustrates the distance, here 8.75 feet. Other distances can of course be used also. Since the detector can be placed close to, or right up against the wall, this enables this point source to be located within the room, for example, for calibration. The head can be rotated so that its side opposite the imaging surface is essentially faced against the wall. This allows the entire distance from one wall to be used to obtain that 8.75 feet. This placing an imaging detector against the wall with its imaging surface facing away from the wall produces additional unexpected advantages.

FIG. 7 illustrates another plan view with both imaging detectors 131, 133 located over the patient imaging table 201. In this embodiment, the patient imaging table 201 has been removed, and is replaced by a patient hospital bed 701, which can be wheeled in without crossing the horizontal tracks. The patient may be wheeled in on this hospital bed, and both the detector arms 130, 132 are rotated by 90° relative to the gantry tower 110. This allows the imaging detectors to be placed over a hospital bed 701 that is rolled into place.

The entire movement and structure can be carried out by a controller 150 which can be a computer that automatically determines movement of the various structures and their speeds of movement according to a preprogrammed sequence. A user interface 152 may allow these devices to be controlled remotely for example.

The general structure and techniques, and more specific embodiments which can be used to effect different ways of carrying out the more general goals are described herein.

Although only a few embodiments have been disclosed in detail above, other embodiments are possible and the inventors intend these to be encompassed within this specification. The specification describes specific examples to accomplish a more general goal that may be accomplished in another way. This disclosure is intended to be exemplary, and the claims are intended to cover any modification or alternative which might be predictable to a person having ordinary skill in the art. For example, other kinds of medical imaging systems could be used with this embodiment. Other differences between the scans could be used. Other compensations can be applied to the scans.

Also, the inventor intend that only those claims which use the words “means for” are intended to be interpreted under 35 USC 112, sixth paragraph. Moreover, no limitations from the specification are intended to be read into any claims, unless those limitations are expressly included in the claims. The computers described herein may be any kind of computer, either general purpose, or some specific purpose computer such as a workstation. The computer may be an Intel (e.g., Pentium or Core 2 duo) or AMD based computer, running Windows XP or Linux, or may be a Macintosh computer. The computer may also be a handheld computer, such as a PDA, cellphone, or laptop.

The programs may be written in C or Python, or Java, Brew or any other programming language. The programs may be resident on a storage medium, e.g., magnetic or optical, e.g. the computer hard drive, a removable disk or media such as a memory stick or SD media, wired or wireless network based or Bluetooth based Network Attached Storage (NAS), or other removable medium or other removable medium. The programs may also be run over a network, for example, with a server or other machine sending signals to the local machine, which allows the local machine to carry out the operations described herein.

Where a specific numerical value is mentioned herein, it should be considered that the value may be increased or decreased by 20%, while still staying within the teachings of the present application, unless some different range is specifically mentioned. Where a specified logical sense is used, the opposite logical sense is also intended to be encompassed.

What is claimed is:

1. A gantry camera, comprising:
a first imaging detector, having an imaging surface;
a first gantry arm, holding said first imaging detector;
a gantry tower, extending substantially vertical, and holding said first gantry arm;
a support, holding said gantry tower, and said support extending substantially horizontally, said support having a moving part allowing said gantry tower to move in a horizontal direction, while the tower is maintained in said vertical direction; and
a first movement stage, which rotates said gantry tower in a vertical plane, relative to said support.

2. A detector as in claim 1, further comprising a second movement stage, causing movement between said gantry arm and said first imaging detector that rotates said first imaging detector relative to said gantry arm by a specified rotation amount.

3. A detector as in claim 2, wherein said second movement stage moves said imaging detector between at least a first orientation which is parallel to said support, and a second orientation which is parallel to a wall that extends perpendicular to said support.

4. A detector as in claim 3, further comprising at least one collimator holder mounted on said wall, and said first movement stage allows moving said gantry arms to place a collimator in said collimator holder, and to remove a collimator from said collimator holder.

5. A detector as in claim 4, wherein there are at least two collimator holders, on said wall, one vertically above the other.

6. A detector as in claim 4, wherein said collimator holders are fixed on said wall and do not swing relative to said wall.

7. A detector as in claim 1, further comprising a controller that controls said first movement stage to rotate said gantry tower by 90° to allow said gantry arm to move in a direction which allows a hospital bed to be inserted underneath without crossing said support.

8. A detector as in claim 1, further comprising a second imaging detector, and a second gantry arm, said second imaging detector and second gantry arm connected to said gantry tower and moved relative thereto.

9. A detector as in claim 2, further comprising a controller that controls said second movement stage to allow said imaging detector to be placed against the wall with its imaging surface facing away from the wall.

10. A detector as in claim 1, wherein said first movement stage allows moving said detector in a direction away from said patient.

11. A method, comprising:
imaging a patient with an imaging detector mounted on a gantry arm, which imaging detector has a collimator located therein;
moving, at a different time than said said imaging, said imaging detector to a second position where an imaging surface of the imaging detector is oriented at a different angle than an angle at which said patient has been imaged;
in said second position, moving said imaging detector away from said patient, to face to a wall which is holding at least one collimator on a collimator holder; and
coupling a collimator which was used for said imaging into a collimator holder that is located on the wall, where said collimator holder holds said collimator in said position which is substantially parallel to said wall.

12. A method as in claim 11, further comprising operating without said collimator holders swinging relative to said wall.

13. A method as in claim 11, further comprising, at another time, rotating the gantry arm to move in a direction which allows a hospital bed to be inserted underneath said gantry arm without crossing a support.

14. A method as in claim 11, further comprising controlling said imaging detector to be placed against the wall with its imaging surface facing away from the wall.

15. A method as in claim 14, further comprising calibrating said imaging detector in said position placed against the wall with its imaging surface facing away from the wall.

16. A method as in claim 11, wherein said moving comprises moving the collimator to a first position and to a second position, where said second position is vertically above said first position.

17. A method comprising:
using a gantry system to image a patient;
allowing the gantry system to be rotated away from the patient and rotating the imaging heads toward a wall;
allowing changing collimator parts on the gantry system automatically, while keeping said imaging heads substantially parallel to the wall.

18. A method as in claim 17, further comprising allowing calibrating said imaging heads while keeping said imaging heads substantially parallel to the wall.
